(19) United States
(12) Patent Application Publication
(10) Pub. No.: US 2017/0319878 A1
(43) Pub. Date: Nov. 9, 2017

(54) PROTECTIVE HOOD

(71) Applicant: SCALDIS ST-MARTIN, Peruwelz (BE)

(72) Inventor: Vincent VANNESTE, Thimougies (BE)

(21) Appl. No.: 15/528,838

(22) PCT Filed: Nov. 26, 2015
(86) PCT No.: PCT/EP2015/077753

§ 371 Date: May 23, 2017

(30) Foreign Application Priority Data
Nov. 28, 2014 (EP) 14195539.3

(51) Int. Cl. A62B 17/04; A62B 17/00; A61F 9/02

(52) U.S. Cl. CPC A62B 17/04; A61F 9/027; A62B 17/001

(57) ABSTRACT

A head protection hood (1) being an integral part of protective clothing (8) for clean rooms, the said hood (1) presents:
An external face (2) designed to be in contact with the surrounding environment,
An internal face designed to be in contact with the user's head,
A section for passing the head through (3),
A facial opening (4) at user eye level, and
A first and second means of connecting (5) safety glasses (L), the said connection means (5) being fastened to the hood (1) at either side of the facial opening (4).

The said hood includes, once in its package safety glasses (L) fastened to the hood (1) by at least one of the said first and second means of attachment.

The present invention concerns a head protection hood being an integral part of protective clothing in clean rooms, the said hood presents:

An external face designed to be in contact with the surrounding environment,

An internal face designed to be in contact with the user's head,

A section for passing over the head,

A facial opening at user eye level, and

A first and second means of connecting safety glasses, the said connection means being fastened to the hood at either side of the facial opening.

Such a hood is known in document FR2 928 260 where the straps that allow the initial adjustment of the hood for the user's head (by tying the straps behind the head) are also used for passing through the rings on the safety glasses when dressing. These straps, according to a previous document, constitute therefore the means of connection, used when dressing to fasten the safety glasses (itself being an individual piece of equipment) to the hood of a combination for use in clean room.

However, the process for fixing the safety glasses to the hood of the combination according to the previous document, even if it minimises the number of times the user touches the glasses, does not reduce the risk of the glasses falling when the user passes the straps through the rings on the glasses. Furthermore, when fixing the glasses, the straps must then be tied at the back of the head, which is not easy. More importantly, if by accident or due to a handling error, the user lets go of the straps or if these latter slip through his hands, there is a strong possibility that the glasses fall to the floor. The result is contamination of this latter, which is intolerable in the domain of clean rooms, and the dressing procedure must be started again, at least partially, at least concerning positioning the glasses.

This type of hood is especially used in aseptic and sterile environments where the user must be isolated from the surrounding environment. This is especially indicated in environments such as clean rooms and laboratories where vaccines and drugs are prepared. On one hand, in this type of environment, it is essential to isolate as best as possible the user in order that he does not contaminate the environment in which he works and, on the other hand, it is often necessary to isolate the user himself from dangerous or toxic substances.

When an operator is required to work in a sterile environment, he must pay particular attention not to contaminate the equipment that he wears.

Thus, the operator must respect the strict dressing procedure, which only make sense if the protective equipment that is used is itself completely sterile. To this end, the combinations and hoods are generally supplied after sterilisation, in sealed packages with easy opening allowing the equipment to be handled.

In order to protect the operator's eyes, the hoods actually used are fitted with a fixed or moveable visor which assures the isolation and protection for the user but which also minimises the risk of contamination of the surrounding environment (vaccines, drugs, . . . ) (see example of protective hood E5 111 101 commercialised by Protextyl®). Nevertheless, several inconveniences are inherent to hoods fitted with a visor. In fact, the presence of a visor restricts the field of view (presence of blind spot) and the formation of condensation on the visor. For this reason, recourse to protective spectacles is the preferred choice at present.

This approach to using safety glasses consists usually of using hoods that are an integral part of a clean room combination, over which are fitted protective masks “ski masks” (goggles) (for example those described in document EP 0 195 517). These safety glasses therefore constitute an individual piece of equipment that the operator must handle with extreme care so as not to let them fall, in which case he would need to change the safety glasses because they had been contaminated. Furthermore, correctly positioning these safety glasses is not easy as it usually involves positioning an elastic band around the operator's head. The operator must therefore stretch the elastic band, pass the glasses over his head and then proceed with the adjustment of the safety glasses in front of his eyes.

All these stages for fitting the safety glasses increase the risk of error in manipulation (dropping the safety glasses, contaminating the front of the goggles, . . . ) and consequently risk of contamination. Note that as indicated above concerning document FR 2 928 260, this same problem is observed for safety glasses the positioning of which involves tying a strap behind the head, this operation being more restrictive than the case of an elastic band, the risks of dropping and contamination being even more important.

It is clear therefore, that at the present time, recourse to safety glasses as a means of protecting the eyes, even if it remains the preferred means, is restrictive, requires several manipulations of the glasses and the attachment system (elastic band, straps, . . . ), slows down the dressing procedure and considerably increases the risk of contamination. In fact, the probability of the safety glasses falling is present and the numerous manipulations required for fitting them increases in a non-negligible manner the risk of transferring contaminants from the operator to the safety glasses and then to the surrounding environment. Furthermore, this approach to the technique generally implies separate sterile packaging for the hood and the safety glasses, these two elements not being subject to the same sterilisation conditions. The operator must therefore, open two different packets, which again, increases in a non-negligible manner the risk of contaminant transfer.

There exists therefore a real need to simplify use of current hoods so as to be an integral part of a combination for clean rooms and used in association with “ski mask” type safety glasses, in order that the steps for covering equipment (clothing) and safety glasses are less restrictive, quicker and more sure in order to minimise any risk of contamination.

The invention aims to address the drawbacks of the technique by procuring a hood being an integral part of the protective clothing for clean room, the said hood allowing to simplify the positioning and use of the safety glasses, in such a manner as to render the positioning of these latter simple and quick, by minimising the handling by the operator who wears the head protection hood whilst assuring a minimisation of contamination risk for both operator and the surrounding environment.

To solve this problem, it is planned following the invention, a hood as indicated at the start, characterised in that, once in its package, and during dressing, the safety glasses (L) are fastened to the hood by at least one of the said first and second means of attachment.

According to the invention, at least one means of attachment is fastened to the hood at each side of the facial opening, this being at least a means of attaching the safety glasses to the hood when this latter, being an integral part of the protective clothing for clean room, is packaged (i.e. Before dressing and in its sterile packaging) but also during dressing (i.e. When putting the combination on). For example, at least one means of attaching the safety glasses to the hood by assuring a link between the hood and the frame of the safety glasses.

So, due to the presence of the means of attachment to either side of the facial opening and that the glasses are held by these means of attachment from the start (i.e. when the combination and therefore the hood, already being an integral part are in the sterile package before use), the operator who wears the protective clothing (combination) and therefore the hood according to the invention does not have to worry about holding the safety glasses. The operator has his hands free and may correctly position the hood over his head without worrying about the safety glasses which are held and therefore there is no risk of them falling.

In addition, when the safety glasses are held by the means of attachment after packaging the combination and therefore the hood in a sterile package, the operator does not have to hold the safety glasses to bring them up to face level and more particularly in front of the hood's facial opening, which eliminates one of the sensitive dressing stages during which it is easy to become contaminated and this therefore reduces the risk of contamination. In fact, if the glasses are held on the hood from the outset, i.e. Before the operator has taken the hood, he does not have to open another sterile package which only contains the glasses and he does not have to follow a complex dressing procedure which consists of passing an elastic band around his head or tying a knot behind his head to assure the position and supporting the safety glasses, these operations being conducive to contamination.

According to the invention, the hood fitted with the attachment means on either side of the facial opening reduces the number of dressing stages and also allows to package the hood (being an integral part of protective clothing for clean room) and the safety glasses in the same sterile package, the glasses being already correctly positioned on the hood, all this contributing to significantly minimise the risk of contamination.

Advantageously, according to the invention, the first means of attachment and/or a second means of attachment connected to the said hood are situated on an axis that traverses horizontally the said facial opening. This positioning for each means of attaching the hood at either side of the facial opening allows the safety glasses to be held and positioned in such a way as to minimise handling and changing position of the safety glasses whilst ensuring correct placement in such a manner as to cover the facial opening and protect the operator's eyes. This position on an axis which traverses horizontally the facial opening also contributes to minimising and simplifying the dressing procedure and consequently the risk of contamination.

For preference, according to the invention, the said first means of attachment and/or a second means of attachment are fastened to the said external face or the said internal face of the hood. When the means of attachment is fastened to the external face of the hood, it may for example be sewn but may also be attached to the hood by any other suitable means. Attachment to the internal face of the hood is also possible according to the invention but, in this case, a slit should be provided close to the lateral edges of the facial opening so that the means of attachment may pass through and find itself at least partially outside the hood.

For preference, according to the invention, the said first means of attachment and/or a second means of attachment are fastened to the said external face or the said internal face of the hood.

For preference, according to the invention, the said first means of attachment and/or a second means of attachment present a first extremity attached to the said hood and a second free extremity.

It is in fact possible to fasten the means of attachment either at the back of the hood, or on the lateral part of the hood. In both cases, a first extremity of the means of attachment is fastened to the hood whereas only the second free extremity is arranged to be situated laterally in relation to the facial opening.

Preferentially, according to the invention, the first means of attachment and/or a second means of attachment include an engagement element to be engaged in a corresponding engagement element fastened to the safety glasses.

Advantageously, according to the invention, the said engagement element includes a male extremity or female so arranged to engage in a corresponding engagement element including a corresponding male or female extremity fastened to the safety glasses.

All attachment means fastened to the hood which allow the safety glasses to be held, for example by the frame of the latter, may be envisaged according to the present invention. For example, it may be a system of loops or more simply an elastic.

For preference, according to the invention, the said fastening means is adjustable in length. Adjusting the length of the attachment means ensures suitable positioning of the safety glasses in front of the facial opening in such a manner that the safety glasses closely fit the operator's face and/or the edge of the facial opening, which isolates the operator from the surrounding environment in an optimal manner. For example, if the attachment means is a system of loops, pulling on the free part of the attachment means passing through this system of loops allows the position of the safety glasses to be adjusted.

Advantageously, the hood according to the invention includes an auto-adjustment system for the hood size, positioned at the back of the hood, for example the press-studs allowing the width of the hood and/or height of the hood to be adjusted. By choosing to fit a press-stud on one of the attachments rather than another, it is possible to define a hood size suited to the user's morphology in such a manner that the hood correctly fits the operator's head and is positioned so as not to hinder the operator.

Other systems of auto-adjustment such as straps or connectors may also allow the hood to be adjusted to the operator's morphology. Consequently, a hood made according to the invention may suit several users who may adjust the size. It is obvious that any other suitable means of auto-adjustment may be used within the scope of the present invention.

For preference, the hood made according to the invention is an integral part of the protective clothing which is a combination as divulged in document EP2 303 044; this combination includes an external face designed to be in contact with a surrounding environment and an internal face treated to be in contact with the handler; a first and second section of leg fastened to each other via an opening between the legs (this opening being designed to be closed in such a way that area between the legs presents two closed leg sections); a first and second sleeve and a body part to which are fastened the first and second sleeves at one end, and the first and second leg sections, the said part of the body includes a section to pass the head through, in addition the said combination includes at least one handling zone in contact with the internal face of the body part.

The said handling zone of such a combination may be, for example, a loop, an elastic, tab or cord or may even be formed by an area of tissue (which need not be formally limited) of the combination, this zone being treated for being gripped and held.

Advantageously, the head protection hood, according to the invention is an integral part of protective clothing being itself a combination, the opening of which is situated in the body part, for example at the centre of the thorax or from the shoulder down to the operator's hip.

Other forms of making the hood according to the invention are indicated in the attached claims.

The present invention also relates to the ensemble including:

A head protection hood according to the invention being an integral part of protective clothing for clean room, and

safety glasses held by at least one of the first and second means of attachment fastened to the said hood, when packaged and during dressing.

Other forms of making the ensemble according to the invention are indicated in the attached claims.

The present invention also relates to the process for dressing using clean room protective clothing fitted with a head protection hood according to the invention including the following stages:

Putting on, by the user, of clean room protective clothing by an opening in this latter, with positioning of the user's head inside the said hood, safety glasses fastened to the hood by at least one of the said first and second means of attachment, and

An adjustment, by the user, of the position of the said safety glasses in front of the said facial opening at eye level.

Advantageously, the process according to the invention also includes a stage for attaching the safety glasses to the hood by a second attachment means when the safety glasses are only previously attached to the hood by the first attachment means.

For preference, the process according to the invention includes an additional stage for closing the said opening of the clean room protective clothing, for example by using a zip or any other suitable means of closing.

Other forms of carrying out the process according to the invention are indicated in the attached claims.

Other characteristics, details and advantages of the invention emerging from the description given below, including without limitation and referring to the attached drawings.

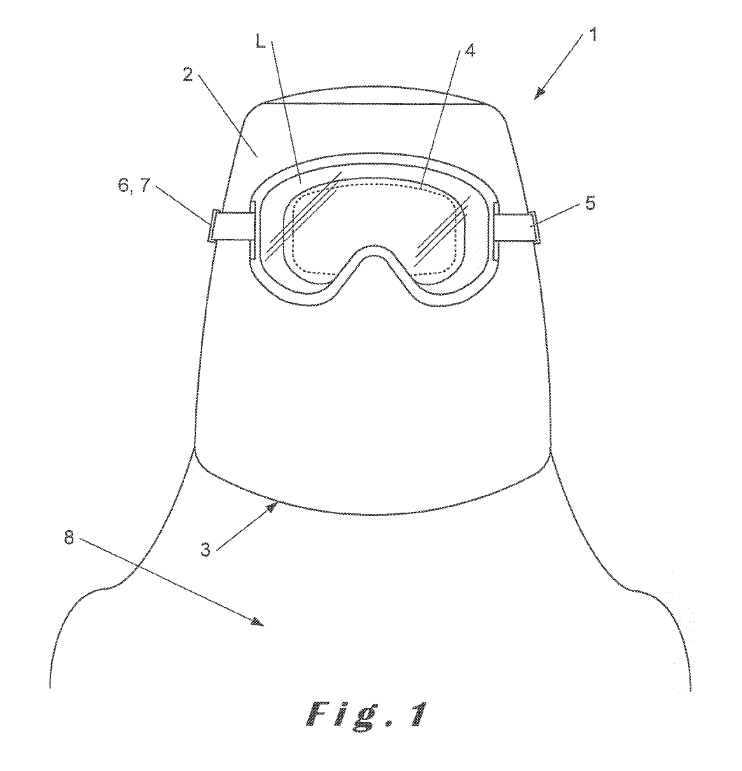

FIG. 1 is a view of the front of the head protection hood being an integral part of the protective clothing according to the invention.

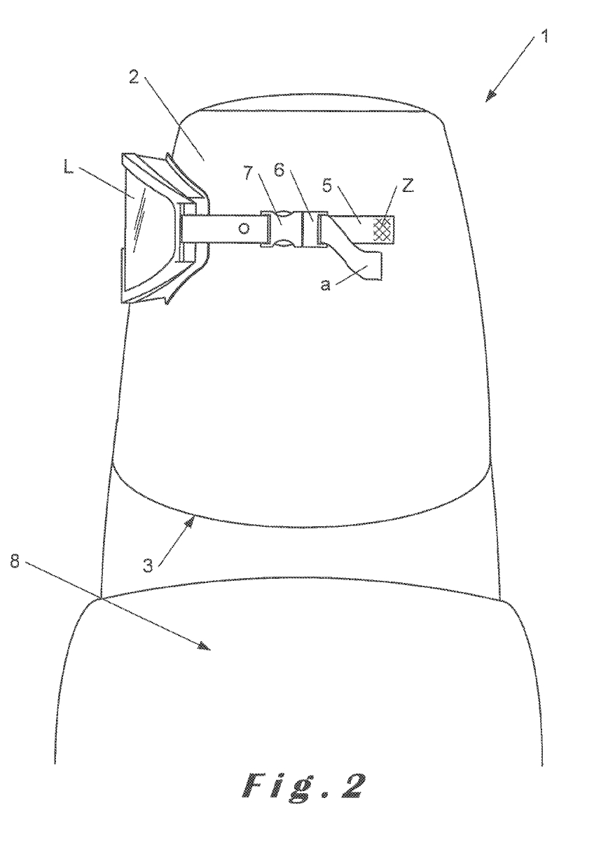

FIG. 2 is a view of the side of the head protection hood being an integral part of the protective clothing according to the invention.

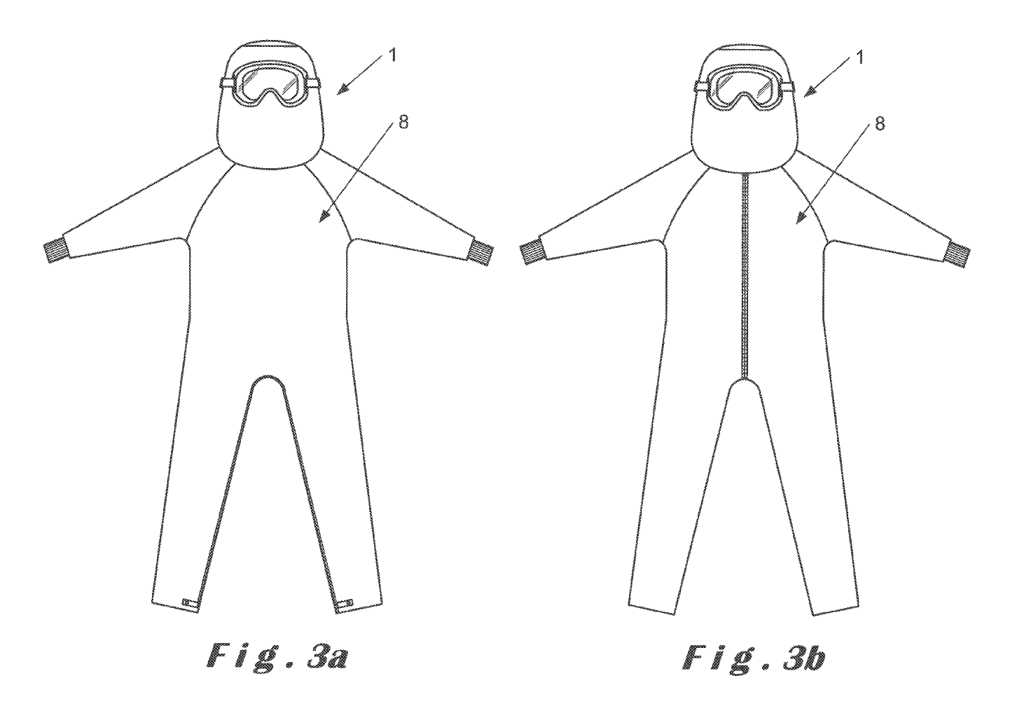

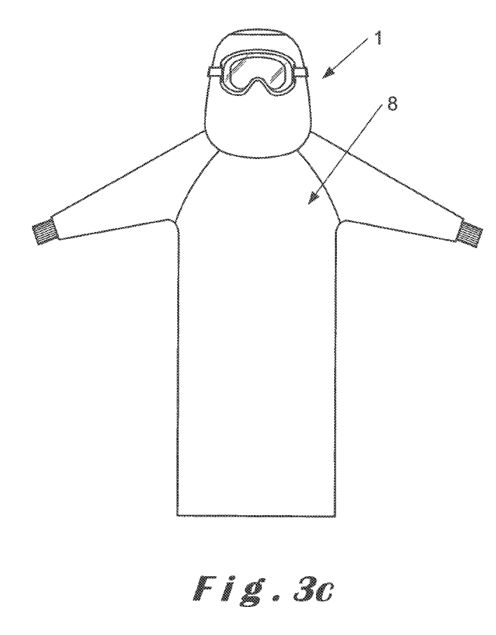

FIGS. 3a, 3b and 3c illustrate a hood according to the invention which is attached and being an integral part of the protective clothing.

In the figures, the identical or analogous elements have the same reference.

FIG. 1 illustrates a hood 1 according to the invention being an integral part of the protective clothing 8 for clean room and which presents an external face 2 planned to be in contact the the surrounding environment, an internal face planned to be in contact with the user's head, a section to pass the head through 3 and a facial opening 4 on a level with the user's eyes. In addition, as shown in FIG. 2, the hood 1 includes, at either side of the facial opening 4 at least one means of attachment 5 for holding the safety glasses L. This means of attachment 5 presents one end z fastened to the hood 1 and a free end which has an engagement element 6 (here is shown a “male” type element). This engagement element 6 is arranged to allow the alignment of a corresponding engagement element 7 (here is shown a female type) present at the safety glasses level L.

In FIG. 1, the engagement element 7 present at the level of the safety glasses L is attached to the frame of the safety glasses by means of a link. It is obvious that this means of attachment is not essential and that the engagement element may be fitted directly to the frame without there being any need for an attachment link.

Furthermore, the means of attachment 5 is adjustable in length, which allows the safety glasses to be brought into contact with the edges of the facial opening 4 and/or in contact with part of the operator's face, in such a way that this latter is adequately protected around the eyes. Here, the adjustment of the length of the attachment means is done by pulling on the free end of the attachment means which passes through the engagement element 6. This is typically the case when the engagement element 6 is in the form of a loop system. It is obvious that any other suitable type of engagement means falls within the scope of the present invention.

Where appropriate, the hood 1 being an integral part of the protective clothing 8 for clean room, may present one or several zones formed from material different to that of the external face of the hood 1, for example in a permeable material. Such zones may be situated either below or above the facial opening 4 in order to allow the operator to breathe easily and also to aerate the interior of the hood 1.

FIGS. 3a, 3b and 3c illustrate a hood 1 which is connected to and is an integral part of the protective clothing 8, here for example, a combination for clean room (FIGS. 3a and 3b) and similar (jacket or gown in

FIG. 3c). In FIG. 3b, the zip is represented on a level with the operator's thorax but it can also be situated at the back of this latter.

It is obvious that the hood 1 may be attached by any suitable means of attachment to the protective clothing 8 in such a manner as to be an integral part, for example by being sewn or attached using a zip, press-studs or even Velcro®.

It is obvious that the present invention is not limited to the forms described above and that modifications may be made whilst remaining within the scope of the attached claims.

1. A head protection hood (1) being an integral part of protective clothing (8) for clean rooms, the said hood (1) presents:
An external face (2) designed to be in contact with the surrounding environment,
An internal face designed to be in contact with the user's head,
A section for passing the head through (3),
A facial opening (4) at user eye level, and
A first and second means of connecting (5) safety glasses (L), the said connection means being fastened to the hood (1) at either side of the facial opening (4).
The said hood (1) being characterised in that, once in its package, and during dressing, the safety glasses (L) are fastened to the hood by at least one of the said first and second means of attachment.

2. The head protection hood (1), according to claim 1 is characterised in that the first means of attachment and/or a second means of attachment (5) connected to the said hood (1) are situated on an axis that traverses horizontally the said facial opening (4).

3. The head protection hood (1) according to claim 1, is characterised in that the said first means of attachment and/or a second means of attachment are fastened to the said external face (2) or the said internal face of the hood (1).

4. The head protection hood (1) according to claim 1, is characterised in that the said first means of attachment and/or a second means of attachment (5) are fastened to the rear part of the said hood (1) or to a lateral part of the said hood (1).

5. The head protection hood (1) according to claim 1, is characterised in that the said first means of attachment and/or a second means of attachment (5) presents a first extremity (z) attached to the said hood (1) and a free second extremity (a).

6. The head protection hood (1) according to claim 1, characterised in that the first means of attachment and/or a second means of attachment (5) include an engagement element (6) so arranged as to be engaged in a corresponding engagement element (7) fastened to the safety glasses (L).

7. The head protection hood (1) according to claim 6, characterised in that the said engagement element (6) includes a male extremity or female so arranged to engage in a corresponding engagement element (7) including a corresponding male or female extremity fastened to the safety glasses (L).

8. The head protection hood (1) according to claim 1, characterised in that at least one means of attachment (5) is adjustable in length.

9. The head protection hood (1) according to claim 1, characterised in that the said hood (1) includes a system of auto-adjustment of the size of the hood (1) positioned at the back of the hood (1).

10. The ensemble includes:
A head protection hood (1) for the head being an integral part of protective clothing (8) for clean rooms, according to claim 1 and
Safety glasses (L) held by at least one of the first and second means of attachment (5) fastened to the said hood (1) when packaged and during dressing.

11. Process for dressing in protective clothing (8) for clean room fitted with a protective hood for the head according to claim 1 of the said process including the following stages:
Putting on, by the user, of clean room protective clothing (8) by an opening in this latter, with positioning the users head inside the said hood (1), safety glasses (L) fastened to the hood (1) by at least one of the first and second means of attachment (5), and
An adjustment, by the user, of the position of the said safety glasses (L) in front of the said facial opening (4) at eye level.

12. Dressing process according to claim 11 includes a stage for attaching the safety glasses (L) to the hood (1) by a second attachment means (5) when the safety glasses (L) are only previously attached to the hood (1) by the first attachment means (5).

13. Process according to claim 11, includes an additional stage for closing the said opening of the clean room protective clothing (8), for example by using a zip or any other suitable means of closing.
